(19) United States
(12) Patent Application Publication
(10) Pub. No.: US 2011/0313399 A1
(43) Pub. Date: Dec. 22, 2011

(54) INTRAVENOUS CANNULA

(75) Inventors: Yuval Jacoby, Tel-Aviv (IL); Adi Alphandary, Hod-HaSharon (IL); Nitzan Bichacho, Tel-Aviv (IL)

(73) Assignee: Vein Flow Inc., Cambridge, MA (US)

(21) Appl. No.: 13/148,486

(22) PCT Filed: Feb. 9, 2010
(86) PCT No.: PCT/IB10/50593

§ 371 Date: Aug. 9, 2011

Related U.S. Application Data
(60) Provisional application No. 61/150,809, filed on Feb. 9, 2009.

(51) Int. Cl. A61M 25/00

(52) U.S. Cl. 604/506; 604/523

(57) ABSTRACT

An intravenous (IV) cannula comprising an elongated body including a distal section for insertion into a blood vessel; at least one channel extending along at least a portion of a longitudinal axis of said cannula section, the at least one channel configured to maintain an amount of at least 10% of a blood flow in the blood vessel; and a central lumen configured to allow an IV fluid flow into the blood vessel.

### RELATED APPLICATION/S

This application is related to U.S. Provisional Application No. 61/150,809 titled “Provisional Patent Application—Channeled Cannula” filed on 9 Feb. 2009, and on which this application claims priority.

The contents of all of the above documents are incorporated by reference as if fully set forth herein.

### FIELD AND BACKGROUND OF THE INVENTION

The present invention, in some embodiments thereof, relates to the field of intravenous infusions and, more particularly, but not exclusively, to an intravenous catheter for allowing sufficient blood flow through a collapsed blood vessel.

Intravenous infusions are medical procedures routinely performed for delivering fluids, optionally including medication, into a patient's bloodstream. Generally, the infusion includes one of two methods, a first method including inserting a needle into a vein and passing the fluid through the needle into the vein, and a second method including inserting a relatively thin cannula into the vein and passing the fluid through the cannula into the blood stream. The second method is generally referred to as “IV (intravenous) cannulation”, typically divided into two categories, peripheral venous access, and central venous access.

Peripheral venous access generally includes inserting a short catheter (cannula) into a peripheral vein such as, for example, those found in the arm or the hand. Occasionally, the vein used may include those in a leg or in a foot. An IV cannulation device typically used for peripheral venous access may be a cannula-over-needle device which may include a flexible cannula through which is inserted a metal needle for piercing a hole through the skin and into the vein through which the cannula may be inserted for positioning inside the vein. Generally attached to a proximal end of the cannula, outside the skin, is a hub. To the hub may be attached a needle grip to which the needle is connected, a flashback chamber for receiving a flashback of blood from the vein, an injection port through which medicine may be injected, an IV infusion line, or a syringe, or any combination thereof. Occasionally, the hub may be capped. The hub may include wings to facilitate handling of the IV cannula by a person administering the IV cannulation to a patient, and to better affix the cannula to the patient (for example, by taping the wings to the arm, or in some cases, by suturing the wings to the arm).

Central venous access generally includes inserting a catheter into a large vein, for example, the superior vena cava, the inferior vena cava, or into the right atrium. Central venous access generally serves to deliver the fluid more quickly into the heart from where it may be distributed more quickly throughout the body, in some cases being more advantageous than peripheral venous access. A negative aspect of using central venous access includes difficulty in locating the vein for insertion of the catheter, which typically requires very skilled medical personnel and/or the use of imaging equipment such as ultrasound. Furthermore, due to the direct access to the heart, extreme caution is generally required when inserting the cannula.

Some risks associated with IV cannulation may include chemical irritation due to the contents of the fluid introduced into the vein, which may cause phlebitis, infection, pain, or any combination thereof. Another risk may be thrombophlebitis where a thrombus develops in the infected area. Another risk may be extravasation which may occur due to a partial occlusion in the cannula due to a venous constriction, and which results in a back flow of the fluid into tissue surrounding the area of insertion of the cannula. These risks, among others, may in some cases result in loss of vein functionality and/or possible eventual destruction of the vein. In some extreme cases, they may even result in loss of life.

Generally, an IV cannula is selected of a minimum practical size according to the size of the blood vessel into which it is to be inserted, a length of time during which the cannula is to be inserted in the vessel, and a viscosity of the fluid to be administered. The selection may take into consideration such factors as degree of patient discomfort, fluid flow rate, and ease of insertion. The fluid flow rate is proportional to a diameter of the cannula and a pressure difference across the cannula, and inversely proportional to the length of the cannula and the viscosity of the fluid.

### SUMMARY OF THE INVENTION

There is provided in accordance with an exemplary embodiment of the invention, an intravenous (IV) cannula comprising an elongated body including a distal section for insertion into a blood vessel; at least one channel extending along at least a portion of a longitudinal axis of the cannula section, the at least one channel configured to maintain an amount of at least 10% of a blood flow in the blood vessel; and a central lumen configured to allow an IV fluid flow into the blood vessel.

In an exemplary embodiment of the invention, the at least one channel is formed on an external surface of the cannula portion.

In an exemplary embodiment of the invention, the at least one channel forms at least one lumen between the cannula section and the blood vessel.

In an exemplary embodiment of the invention, a cross-sectional area of a channel is in a range between 0.02 mm-2 mm.

In an exemplary embodiment of the invention, a total cross-sectional area of the at least one channel is at least 10% of a cross-sectional area of the cannula section bounded by a smallest circle. Optionally, a total cross-sectional area of the at least one channel is at least 10% of a cross-sectional area of the cannula section bounded by a smallest ellipse.

In an exemplary embodiment of the invention, the at least one channel is disposed in a straight line. Optionally, the at least one channel is disposed in a helical configuration. Optionally, the helical configuration comprises a helix angle of at least 5°.

In an exemplary embodiment of the invention, a depth of the at least one channel is at least 1.2 times a maximum collapsible distance of a portion of a wall of the blood vessel.

In an exemplary embodiment of the invention, the at least one channel extends along a whole length of the IV cannula.

In an exemplary embodiment of the invention, the blood vessel is a peripheral vein or artery. Optionally, the vessel is a central vein or artery.

In an exemplary embodiment of the invention, the IV cannula comprises apertures for providing multiple exit points in the cannula for the IV fluid. Optionally, the exit points are in the blood vessel. Optionally, the exit points are in subcutaneous tissue.

In an exemplary embodiment of the invention, the lumen is further configured to guide a needle through the cannula.

In an exemplary embodiment of the invention, the channel is formed internally in the cannula.

In an exemplary embodiment of the invention, the cannula section is collapsible.

In an exemplary embodiment of the invention, the at least one channel comprises 2 or more channels. Optionally, the at least one channel comprises 3 or more channels. Optionally, the at least one channel comprises 4 or more channels. Additionally or alternatively, the at least one channel comprises 4 or more channels.

In an exemplary embodiment of the invention, the at least one channel is crescent-shaped. Optionally, the at least one channel is star-shaped. Optionally, the at least one channel is clover-shaped.

In an exemplary embodiment of the invention, the at least one channel is configured to maintain an amount of at least 20% of a blood flow in the blood vessel. Optionally, the at least one channel is configured to maintain an amount of at least 30% of a blood flow in the blood vessel. Optionally, the at least one channel is configured to

maintain an amount of at least 40% of a blood flow in the blood vessel. Optionally, the at least one channel is configured to maintain an amount of at least 60% of a blood flow in the blood vessel.

There is provided in accordance with an exemplary embodiment of the invention, a method of IV cannulation comprising inserting an intravenous (IV) cannula into a blood vessel; administering an IV fluid into the lumen of the cannula; and allowing a portion of the blood vessel to collapse onto the IV cannula and maintaining an amount of at least 10% of a blood flow in the blood vessel.

In an exemplary embodiment of the invention, the method comprises allowing blood flow through at least one channel in the IV cannula. Optionally, the at least one channel extends along a portion of a length of the IV cannula. Optionally, the at least one channel is disposed in a straight line. Optionally, the at least one channel is disposed in a helical configuration. Optionally, the at least one channel forms at least one lumen between the cannula and the collapsed blood vessel.

In an exemplary embodiment of the invention, the method comprises performing said IV cannulation in a peripheral vein or artery. Optionally, the method comprises performing the IV cannulation in a central vein or artery.

In an exemplary embodiment of the invention, the method comprises maintaining an amount of at least 20% of a blood flow in the blood vessel. Optionally, the method comprises maintaining an amount of at least 30% of a blood flow in the blood vessel. Optionally, the method comprises maintaining an amount of at least 50% of a blood flow in the blood vessel. Optionally, the method comprises maintaining an amount of at least 60% of a blood flow in the blood vessel.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1A schematically illustrates an exemplary cannula-over-needle device including a channeled cannula inserted in a blood vessel, according to an embodiment of the present invention;

FIGS. 1B-1D schematically illustrate exemplary distal sections of the channeled cannula of FIG. 1A, according to some embodiments of the present invention;

FIGS. 2A-2F schematically illustrate exemplary cross-sectional views A-A of the channeled cannula in FIG. 1, according to some embodiments of the present invention;

FIGS. 3A-3C schematically illustrate exemplary cross-sectional views B-B of the channeled cannula in FIG. 1 including a needle inside a central lumen, according to some embodiments of the present invention;

FIGS. 3D and 3E schematically illustrate an exemplary cross-sectional view of the channeled cannula of FIG. 1 in an exemplary embodiment of a collapsible cannula prior to removal of a needle, and following removal of the needle, according to some embodiments of the present invention;

FIG. 4A schematically illustrates a perspective view of a cannula portion including four channels linearly disposed along a length of the portion, and a perspective view of the cannula portion inside a blood vessel, according to some embodiments of the invention;

FIG. 4B schematically illustrates a perspective view of a cannula portion including three channels helically disposed along a length of the portion, and a perspective view of the cannula portion inside a blood vessel, according to some embodiments of the invention;

FIG. 4C schematically illustrates a perspective view of a cannula portion including a single channel linearly disposed along a length of the portion (crescent shaped channel), and a perspective view of the cannula portion inside a blood vessel, according to some embodiments of the invention;

FIG. 5A-5C schematically illustrate exemplary cannula portions inserted through skin into a blood vessel, according to some embodiments of the present invention;

FIGS. 5D-5F schematically illustrate exemplary cross-sectional views C-C of the channeled cannula in FIG. 5A, according to some embodiments of the present invention;

FIG. 6A schematically illustrates a perspective view of a cannula portion including four channels linearly disposed along a length of the portion, according to some embodiments of the invention;

FIG. 6B schematically illustrates a perspective view of a cannula portion including three channels helically disposed along a length of the portion, according to some embodiments of the invention;

FIG. 6C schematically illustrates a perspective view of a cannula portion including a single channel linearly disposed along a length of the portion (crescent shaped cross-sectional channel), according to some embodiments of the invention;

FIG. 7 schematically illustrates an IV cannulation device including a channeled cannula with apertures used in peripheral venous access, according to some embodiments of the invention;

FIG. 8 schematically illustrates an IV cannulation device including a channeled cannula with apertures used in central venous access, according to some embodiments of the invention; and

FIG. 9 illustrates a flow chart of a method for using the exemplary cannula-over-needle device including the channeled cannula shown in FIG. 1, according to an embodiment of the present invention.

### DESCRIPTION OF EMBODIMENTS OF THE INVENTION

The present invention, in some embodiments thereof, relates to the field of intravenous infusions and, more particularly, but not exclusively, to an intravenous (IV) catheter for allowing blood flow through a collapsed blood vessel.

A blood vessel into which an IV catheter has been inserted may collapse unto the catheter (or cannula), interfering with blood flow in the vessel. This may increase a probability of phlebitis and pain in a patient as chemicals in an IV fluid are not rapidly dispersed. Furthermore, a clog may form in the vessel, or an infection may develop (or worsen) as elements of the immune system cannot properly reach a site of the infection.

An aspect of some embodiments of the present invention relates to an IV cannula that maintains a sufficient blood flow through a blood vessel which is at least partially obstructed by a vessel portion which has collapsed onto the cannula. This is achieved by including a channel in the IV cannula which bypasses the collapsed vessel portion and through which the blood may flow. Optionally, the channel includes a plurality of channels. A sufficient amount of blood flow bypassed through the channel may be at least 10% of the blood flow through the vessel prior to collapse of the vessel portion and following insertion of the IV cannula, for example, 15%, 25%, 40%, 55%, 75, 85%, 95%, and 100%. Optionally, a sufficient amount of blood flow may be 10% of the blood flow through the vessel prior to insertion of the IV cannula, for example, 15%, 25%, 40%, 55%, 75, 85%, 95%, and 100%. In some exemplary embodiment s of the present invention, the IV cannula includes an elongated body including a distal section (referred to hereinafter as cannula section or cannula portion) which may be partly inserted into a blood vessel, and a central lumen configured to allow an IV fluid flow into the blood vessel. The IV cannula may include a cross-sectional shape which is circular. Optionally, the cross-sectional shape is elliptical, star-shaped, triangular , rectangular, crescent-shaped or other polygonal shape, and may include rounded edges.

According to some exemplary embodiments of the present invention, the cannula section includes at least one channel extending along at least a portion of a longitudinal axis of the cannula section. Optionally, the channel includes grooves which extend along at least a portion of the cannula section. Optionally, the cannula section is inserted inside the blood vessel. Optionally, the grooves are formed on an external surface of the cannula section. The grooves may be disposed in a straight line along the length of the cannula section. Optionally, the grooves are disposed helically along the length of the cannula section and include a helix angle of at not less than 5°, for example 15°, 30°, 45°, 60°, 75°, 85°. Optionally, the grooves are disposed in a serpentine configuration (curving shape) along the length of the cannula section. Optionally, the channel may include any shape which does not interfere with vessel blood flow through the channel. Additionally or alternatively, the channel may extend along a whole length of the IV cannula. Optionally, the channel may be located at any point in a periphery of the cannula section. In some exemplary embodiments, the external surface of the cannula section may include a plurality of protrusions, for example resembling bumps, such that channels are formed between the bumps.

According to some exemplary embodiments of the present invention, the channel forms a lumen bordered on one side by the external surface of the cannula section and on an opposing side by collapsed vessel portion, through which the blood flows. The channel is designed so that collapse of the vessel portion does not block the channel. Optionally, only the cross-sectional area of the lumen (and channel) is reduced, Optionally, blood flow through the channel substantially prevents collapse of the portion of the vessel wall.

According to some embodiments of the present invention, a sum of a cross-sectional area of all channels in the cannula section may be in a range of 10% to 55% of a total cross-sectional area of the IV cannula bounded by a smallest circle for example, 10% to 20%, 20% to 30%, 30%-40%, 40%-55%. Optionally, the total cross-sectional area of the IV cannula is measured bounded by a smallest ellipse. Optionally, a cross-sectional area of a single channel may range from 0.02 mm²-2.0 mm². In some exemplary embodiments, a size of the IV cannula may range from 14 gauges (2 mm diameter) to 22 gauges (0.8 mm diameter). Optionally, a depth D of the channel, measured from a perimeter of the smallest circle bounding the IV cannula is at least 1.2×d, where d is a maximum collapsible distance of the vessel portion bordering the lumen. Optionally, the channel may be of any depth D, which may be less than 1.2×d, such that sufficient blood flow is maintained when the vessel portion collapses into the channel.

According to some embodiments of the present invention, the channel may be formed internally in the cannula section. Optionally, the channel includes a first opening through which blood may flow into a central lumen in the IV cannula and flows out together with the IV fluid through an exit point at a distal section. Optionally, a conduit connects the first opening to a second opening through which the blood flows out of the cannula section bypassing the collapsed vessel portion. In some exemplary embodiments, the cannula section includes more than one opening through which the blood may flow into the cannula section, for allowing a different length of cannula section to be inserted into the blood vessel. Optionally, the cannula section may include more than one opening through which the blood may flow out of the conduit. Optionally, the cannula section may include more than one conduits through which the blood may flow.

According to some embodiments of the present invention, the IV cannula may include apertures along the cannula section for delivering the IV fluid into the blood vessel through multiple exit points in addition to the single exit point in the distal section. Optionally, all the fluid flows into the blood vessel through the apertures. Optionally, blood flow is out the exit points. In some exemplary embodiments, the apertures may serve for subcutaneous administration of the IV fluid. Optionally, the cannula section may include a single line of apertures, or a plurality of lines of apertures. For example, the cannula section may have two lines of apertures, three lines of apertures, four lines of apertures, or more. Additionally or alternatively, the apertures are located in the channels.

According to some embodiments of the present invention, the IV cannula may be used as a cannula-over-needle device, configured to allow a needle to be guided through the central lumen. In some exemplary embodiments, the central lumen may include a cross-sectional geometry similar to that of the IV cannula. Optionally, the central lumen may include other cross-sectional geometries suitable for transporting the IV fluid and/or for guiding the needle, for example, a circular cross section or an elliptical cross-section.

According to some embodiments of the present invention, the needle inserted through the central lumen may include a cross-sectional geometry similar to that of the lumen. Optionally, the needle may include channels on an exterior surface for easing introducing of the IV cannula through the skin when pierced by the needle. Optionally, the needle may include other cross-sectional geometries, for example, a circular cross-section. Optionally, the needle may include a solid cross-section. Optionally, the needle cross-section may include a needle lumen. Additionally or alternatively, the needle may include a metal needle.

According to some embodiments of the present invention, the IV cannula partially collapses inwards to form the channel when the needle is removed. Optionally, prior to removal of the needle, the cannula section may include a circular cross-section supported by a round needle. Optionally, the cannula section may include a cross-sectional shape supported by a cross-sectional shape of the needle.

According to some embodiments of the present invention, the IV cannula may be attached to a hub, and may be used for peripheral venous access. Optionally, the IV cannula may be used for central venous access. Connected (affixed, attached) to the hub may be an injection port, wings, valves, needle grip and needle, flashback chamber, bushing, luer connector, luer lock plug, injection port cap, or any combination thereof. Optionally, other components used for IV infusion may be attached. For example, attached to the hub may be an IV infusion line which may be connected to an IV bag. Optionally, a syringe may be attached to the hub. Optionally, the cannula may be made from a biocompatible material which may include polytetrafluoroethylene (Teflon®), or other polymeric and/or non-polymeric materials.

Potential advantages of some exemplary embodiments of the channeled cannula include providing a greater dilution of irritating substances in an increased volume of blood flow; decreasing a likelihood of clotting by reducing the interference to blood flow; and decreasing the likelihood of infection by reducing stasis and increasing the availability of elements of the immune system in the blood to be delivered to the site of infection.

Before explaining at least one embodiment of the invention in detail, it is to be understood that the invention is not necessarily limited in its application to the details of construction and the arrangement of the components and/or methods set forth in the following description and/or illustrated in the drawings. The invention is capable of other embodiments or of being practiced or carried out in various ways.

Before explaining at least one embodiment of the invention in detail, it is to be understood that the invention is not necessarily limited in its application to the details set forth in the following description. The invention is capable of other embodiments or of being practiced or carried out in various ways.

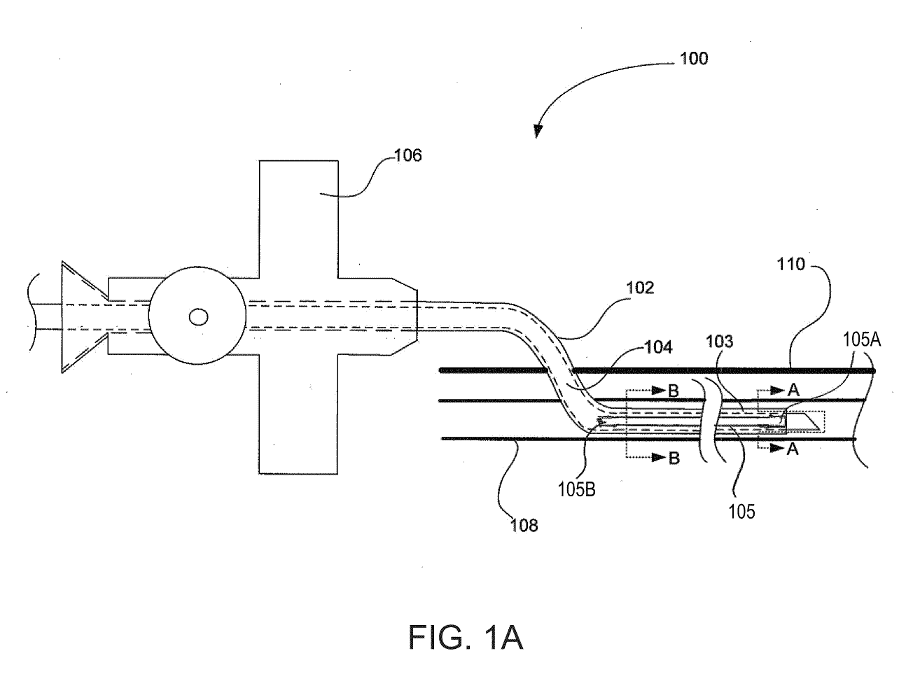

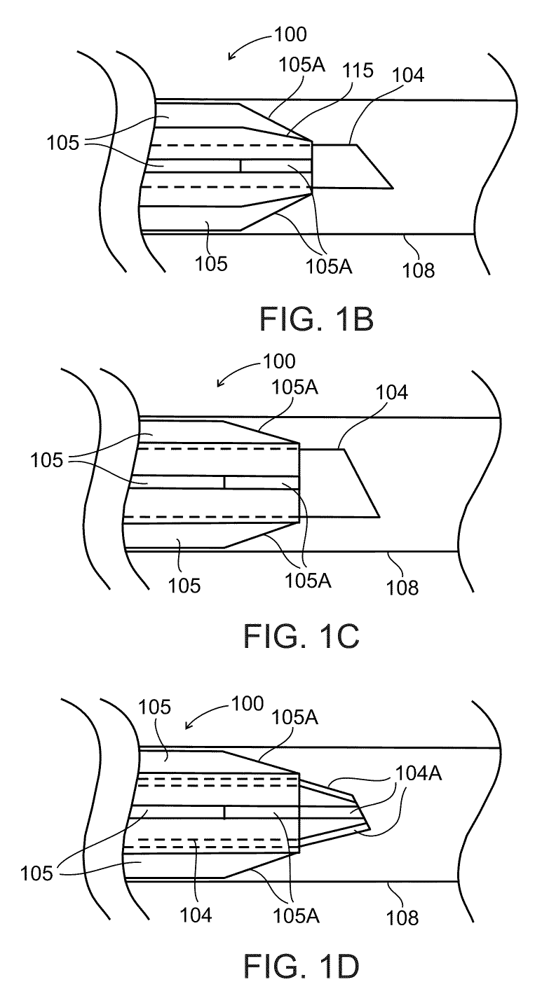

Referring now to the drawings, FIG. 1A schematically illustrates an exemplary cannula-over-needle device 100 including an IV cannula 102 (channeled cannula) inserted in a blood vessel 108, according to an embodiment of the present invention. Device 100 is configured to deliver an IV fluid into blood vessel 108 while maintaining sufficient blood flow in the vessel in case of a vessel potion collapsing onto the device. Device 100 may be used for peripheral venous access and/or central venous access. The IV fluid may include crystalloids, colloids including blood (for example, as in a transfusion), and medication (including drugs). Blood vessel 108 may include a vein or an artery of a human or other living creature (for example, as may be used in veterinary medicine).

Device 100 includes a hub 106 to which a proximal end of cannula 102 is attached, the hub configured to transfer the IV fluid into a central lumen 103 in the cannula for transporting the fluid into blood vessel 108. Hub 106 may include components such as an injection port; wings; valves; a needle grip and a needle, for example needle 104; a flashback chamber; a bushing; a luer connector; a luer lock plug; an injection port cap; or any combination thereof. Optionally, other components used for administering IV fluid and suitable for connecting to a hub as known in the art may be attached to hub 106 such as, for example, an IV line which may connect to an IV bag and/or a syringe.

According to an embodiment of the present invention, cannula 102 is configured to allow sufficient blood flow in vessel 108 to continue while the cannula portion (cannula section) is inside the vessel and the vessel wall portion has collapsed onto the cannula portion. Cannula 102 includes a channel 105 extending from a channel entry point 105A to a channel exit point 105B, and disposed along a length of the cannula section along which vessel blood may flow for bypassing the collapsed wall portion. A sufficient amount of blood flow bypassed through the channel may be at least 10% of the blood flow through the vessel prior to collapse of the vessel portion and following insertion of the IV cannula, for example, 15%, 25%, 40%, 55%, 75, 85%, 95%, 100%.

Reference is now also made to FIGS. 1B-1D which schematically illustrate enlarged views of a distal section of cannula 102, according to some exemplary embodiments of the present invention. FIG. 1B shows cannula 102 with a sloping distal end 115 substantially conforming to sloping channel ends 105A, for allowing easy insertion of the cannula through skin 110 and into blood vessel 108. FIG. 1C shows cannula 102 with sloping channel ends 105A, for allowing easy insertion of the cannula through skin 110 and into blood vessel 108. FIG. 1D shows cannula 102 with sloping channel ends 105A, and needle 104 including sloping channels 104A, for allowing easy insertion of the cannula through skin 100 and into blood vessel 108.

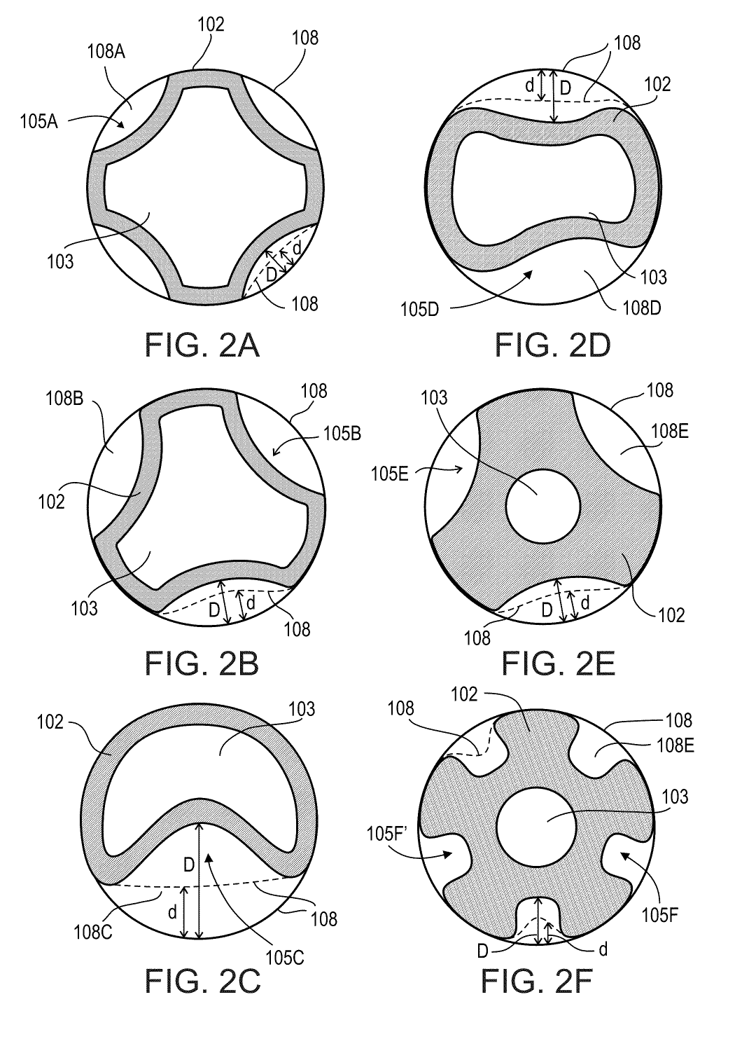

Reference is now also made to FIGS. 2A-2F which schematically illustrate exemplary cross-sectional views A-A of channeled cannula 102 in FIG. 1, according to some embodiments of the present invention.

In an exemplary embodiment of the present invention, FIG. 2A shows a cross-section of cannula 102 inside vessel 108, the cannula portion including four channels 105A extending along the length of the portion, and through which vessel blood may flow. Channel 105A may be similar to that shown in FIG. 1 at 105. Optionally, four channels 105A may extend along a whole length of cannula 102. Each channel 105A forms a lumen 108A extending the length of the channel and bordered on one side by cannula 102 and on an opposing side by a portion of a wall (wall portion) of vessel 108, inside which the vessel blood flows. Optionally, a pressure exerted by the vessel blood flowing inside lumen 108A and through channel 105A substantially prevents the wall portion of vessel 108 from collapsing (inwards in a direction of cannula 102). Optionally, blood flow inside lumen 108A is sufficiently maintained if the wall portion of vessel 108 collapses inwards.

Additionally shown in FIG. 2A is a four leaf clover-shaped cross-sectional view of central lumen 103 which includes a similar geometry to that of cannula 102. Central lumen 103 extends from proximal end of cannula 102 to a distal end of the cannula (openings at both ends) and is configured to transport IV fluid through the cannula into blood vessel 108. Optionally, central lumen 103 is configured to serve as a passageway for guiding needle 104 into skin 110 and into blood vessel 108. Optionally, central lumen 103 may include other cross-sectional geometries suitable for transporting the IV fluid and/or for guiding needle 104, for example, a circular cross-section. Optionally, central lumen may include a plurality of cross-sectional geometries along the length of cannula 102, for example a similar geometric shape as the cannula portion.

In an exemplary embodiment of the present invention, FIG. 2B shows a 3-pointed star-shaped cross-section of cannula 102 inside vessel 108, the cannula portion including three channels 105B extending along the length of the portion and through which vessel blood flows. Channel 105B may be similar to that shown in FIG. 1 at 105. Also shown is central lumen 103 for transporting the IV fluid and/or guide needle 104, the central lumen including a cross-sectional geometry similar to that of the cannula portion; and three lumens 108B in which the vessel blood flows bordered by cannula 102 and the portion of the wall of vessel 108. The exemplary embodiment shown in this figure may be functionally similar to that shown in FIG. 2A with the difference that in this embodiment cannula 102 includes three channels 105B instead of four channels 105A shown in the previous embodiment.

In an exemplary embodiment of the present invention, FIG. 2C shows a crescent-shaped cross-section of cannula 102 inside vessel 108, the cannula portion including one channel 105C extending along the length of the portion and through which vessel blood flows. Channel 105C may be similar to that shown in FIG. 1 at 105. Also shown is central lumen 103 for transporting the IV fluid and/or guide needle 104, the central lumen including a cross-sectional geometry similar to that of the cannula portion; and a single lumen 108C in which the vessel blood flows bordered by cannula 102 and the portion of the wall of vessel 108. The exemplary embodiment shown in this figure may be functionally similar to that shown in FIG. 2A with the difference that in this embodiment cannula 102 includes one channel 105C instead of four channels 105A shown in the previous embodiment.

In an exemplary embodiment of the present invention, FIG. 2D shows a hamburger-shaped cross-section of cannula 102 inside vessel 108, the cannula portion including two channels 105D extending along the length of the portion and through which vessel blood flows. Channel 105D may be similar to that shown in FIG. 1 at 105. Also shown is central lumen 103 for transporting the IV fluid and/or guide needle 104, the central lumen including a cross-sectional geometry similar to that of the cannula portion; and two lumens 108D in which the vessel blood flows bordered by cannula 102 and the portion of the wall of vessel 108. The exemplary embodiment shown in this figure may be functionally similar to that shown in FIG. 2A with the difference that in this embodiment cannula 102 includes two channels 105D instead of four channels 105A shown in the previous embodiment.

In an exemplary embodiment of the present invention, FIG. 2E shows a 3 pointed star-shaped cross-section of cannula 102 inside vessel 108, the cannula portion including three channels 105E extending along the length of the portion and through which vessel blood flows. Channel 105E may be similar to that shown in FIG. 1 at 105. Also shown is central lumen 103 for transporting the IV fluid and/or guide needle 104, the central lumen including a cross-sectional geometry which is circular; and three lumens 108E in which the vessel blood flows bordered by cannula 102 and the portion of the wall of vessel 108. The exemplary embodiment shown in this figure may be functionally similar to that shown in FIG. 2A with the difference that in this embodiment cannula 102 includes three channels 105E instead of four channels 105A shown in the previous embodiment and central lumen 103 is of a different geometry than the cannula portion (in the previous embodiment the geometry is similar).

In an exemplary embodiment of the present invention, FIG. 2F shows a 5-leaf clover-shaped cross-section of cannula 102 inside vessel 108, the cannula portion including four channels 105F extending along the length of the portion and through which vessel blood flows. Channel 105F may be similar to that shown in FIG. 1 at 105. Also shown is a channel 105F′ differently shaped than 105F, and formed by a different shape of the cannula cross-section in that area. Also shown is central lumen 103 for transporting the IV fluid and/or guide needle 104, the central lumen including a cross-sectional geometry which is circular; and five lumens 108F in which the vessel blood flows bordered by cannula 102 and the portions of the wall of vessel 108. The exemplary embodiment shown in this figure may be functionally similar to that shown in FIG. 2A with the difference that in this embodiment cannula 102 includes five channels 105F instead of four channels 105A shown in the previous embodiment and central lumen 103 is of a different geometry than the cannula portion (in the previous embodiment the geometry is similar).

The exemplary embodiments shown above in FIGS. 2A-2F are for illustrative purposes only, and are not intended to be limiting in any way. It should be evident to an ordinary person skilled in the art that there are numerous geometries which may be used for channeled cannula 102, lumen 103, channels 105A-105F, and lumens 108A-108F. Additionally, it should be evident that the distribution of a position of the channels within channeled cannula 102 is for exemplary purposes only, and the there are numerous possibilities as to how the channels may be arranged.

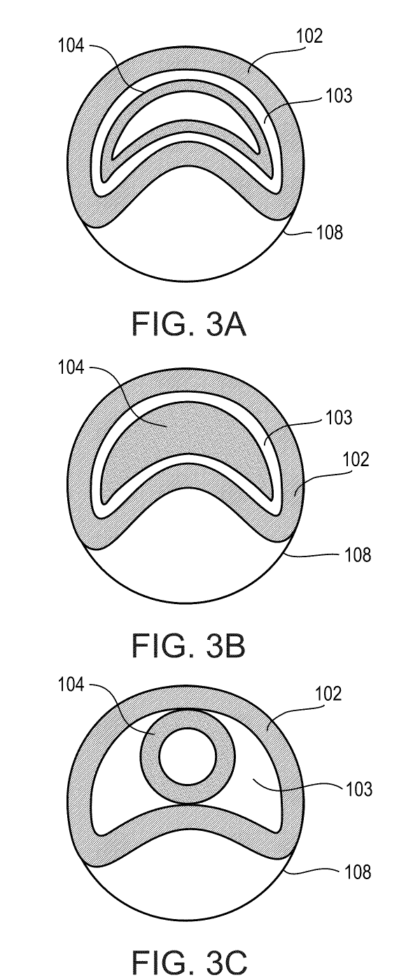

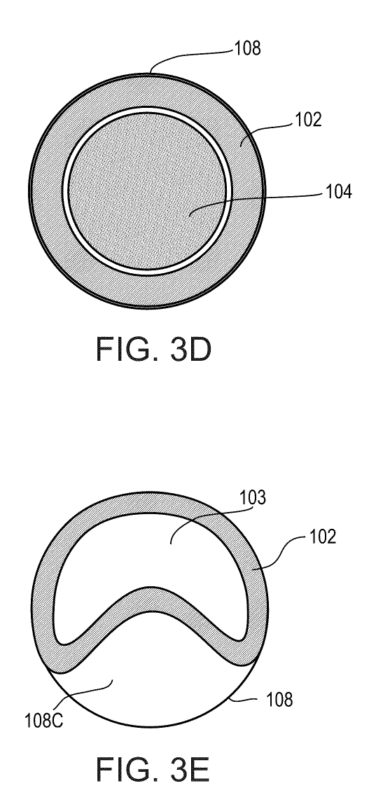

Reference is now also made to FIGS. 3A-3E which schematically illustrate exemplary cross-sectional views B-B of the channeled cannula 102 in FIG. 1 including needle 104 inside central lumen 103, according to some embodiments of the present invention. For illustrative purposes only channeled cannula 102 in FIG. 2C is shown, and is not intended to be limiting in any way. It should be evident to an ordinary person skilled in the art that any of channeled cannula 102 shown in FIGS. 2A-2F may have been used herein. Furthermore, it should be evident that the channeled cannula may include any geometry suitable for insertion in blood vessel 108 and transporting IV fluids, and for including the channels.

In an exemplary embodiment of the present invention, shown in FIG. 3A is needle 104 including a cross-sectional geometry similar to that of the lumen 103 (and cannula 102). Needle 104 includes a hollow interior (a lumen) which may optionally be used to administer medication and/or other types of IV fluids.

In an exemplary embodiment of the present invention, shown in FIG. 3B is needle 104 including cross-sectional geometry similar to that of the lumen 103 (and cannula 102). Needle 104 includes a solid cross-section.

In an exemplary embodiment of the present invention, shown in FIG. 3C is needle 104 including a cross-sectional geometry different from that of lumen 103 (and cannula 102), for example circular as shown. Needle 104 includes a hollow interior (a lumen) which may optionally be used to administer medication and/or other types of IV fluids. Optionally, the cross-sectional geometry of needle 104 may include any other shape suitable for guiding through lumen 103 and for inserting through skin 110 and into blood vessel 108. Optionally, needle 104 may include a solid cross-section.

In an exemplary embodiment of the present invention, shown in FIG. 3D is cannula 102 with circular needle 104 inside, the cannula section configured to partially collapse inwards to form a channel. FIG. 3E shows the cannula section collapsed inwards, forming channel 108C.

A typical mode of operation of device 100 may be described as follow:

Hub 106 is assembled with the different components according to the specific application for which the device is to be used (for example, channeled cannula 102 is selected according to gauge and cross-sectional geometry based on the IV cannulation to be performed; wings may be attached; an IV line may be attached, etc). Needle 104 is inserted through hub 106 and into lumen 103 through the proximal end of channeled cannula 102. Needle 104 is guided through lumen 103 out the distal end and through skin 110 into blood vessel 108. Cannula 102 is then advanced into vessel 108 until the cannula portion is inside the vessel, at which time needle 104 may be removed from vessel 108 and extracted from lumen 103. Cannula 102 is then ready to receive IV fluid which is then transported into blood vessel 108 through lumen 103. Blood flow in vessel 108 is sufficiently maintained by the cannula portion inside the vessel by flowing through channel 105 in cannula 102.

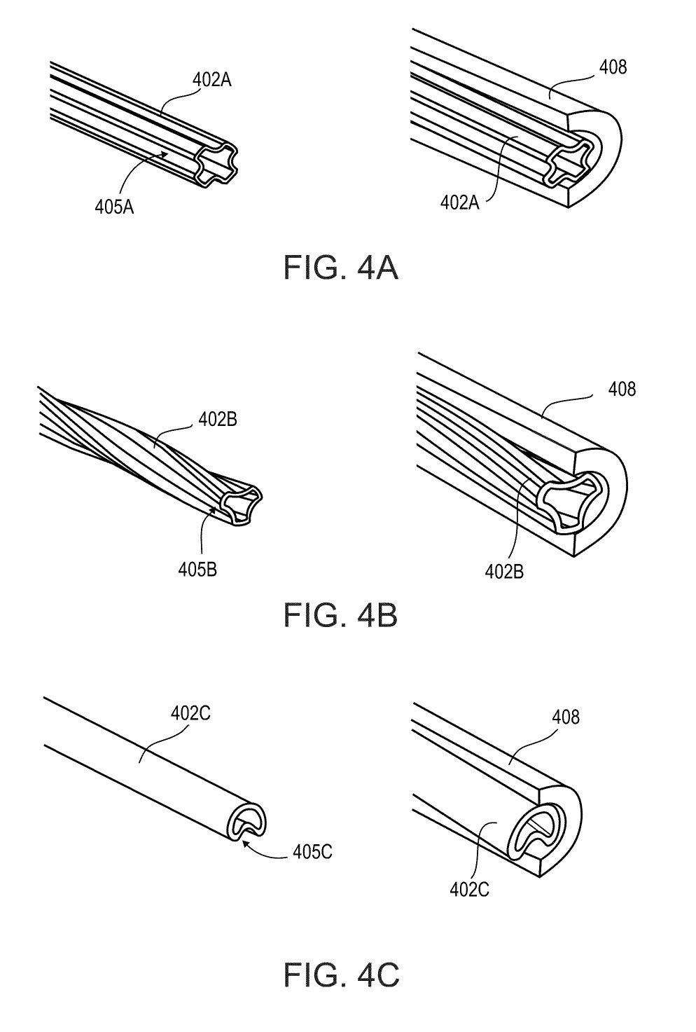

Reference is now made to FIG. 4A which schematically illustrates a perspective view of a cannula portion 402A including four channels 405A disposed in a straight line, and a perspective view of the cannula portion inside a blood vessel 408, according to some embodiments of the invention. Cannula portion 402A, including channel 405A, may be similar to that shown in FIG. 2A at 102, including 105A. Optionally, cannula portion 402A may extend a whole length of the channeled cannula.

Reference is now made to FIG. 4B which schematically illustrates a perspective view of a cannula portion 402B including three channels 405B disposed in a helix, and a perspective view of the cannula portion inside a blood vessel 408, according to some embodiments of the invention. Cannula portion 402B, including channel 405B, may be similar to that shown in FIG. 2B at 102, including 105B. Optionally, cannula portion 402B may extend a whole length of the channeled cannula.

Reference is now made to FIG. 4C which schematically illustrates a perspective view of a cannula portion 402C including a single channel 405C disposed in a straight line (crescent shaped channel), and a perspective view of the cannula portion inside a blood vessel 408, according to some embodiments of the invention. Cannula portion 402C, including channels 405C, may be similar to that shown in FIG. 2C at 102, including 105C. Optionally, cannula portion 402C may extend a whole length of the channeled cannula.

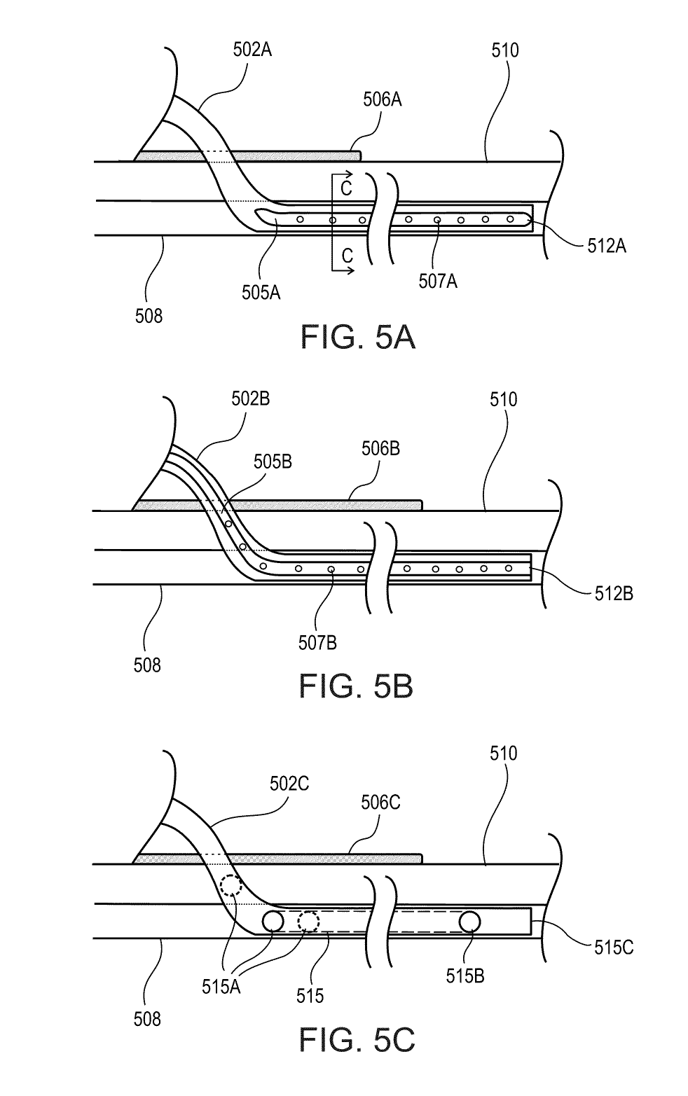

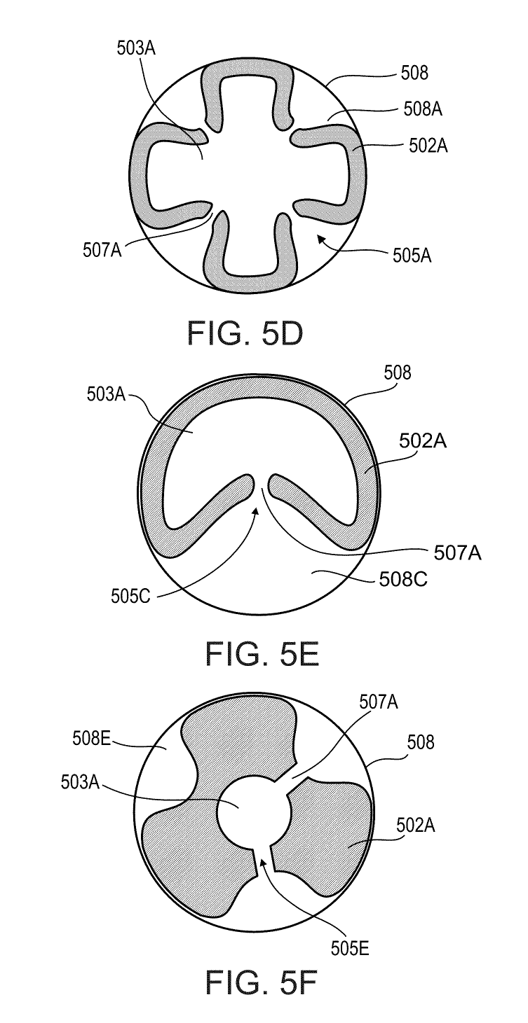

Reference is now made to FIG. 5A which schematically illustrates an exemplary cannula portion 502A inserted through skin 510 into a blood vessel 508, and held in position by a wing 506A, according to an embodiment of the present invention. Cannula portion 502A includes a channel 505A for sufficiently maintaining a blood flow in vessel 508 while the cannula is inside the vessel. Cannula portion 502A additionally includes apertures 507A for delivering IV fluid into vessel 508 through multiple exit points in addition to a single exit point at a distal end 512A of the cannula. Optionally, the IV fluid is delivered only through apertures 507A. Optionally, apertures 507A are arranged in a single line along cannula portion 502A, or in a plurality of lines along the cannula portion. Additionally or alternatively, apertures 507A are included in channel 505A, and may be included in only one channel, or in a plurality of channels. Cannula portion 502A including channel 505A may be similar to cannula 102 including channel 105 shown in FIGS. 1 with the exception of apertures 507A. Reference is now also made to FIGS. 5D-5F which schematically illustrate exemplary cross-sectional views C-C of channeled cannula 502A in FIG. 5A, according to some embodiments of the present invention.

In an exemplary embodiment of the present invention, FIG. 5D shows a cross-section of cannula 502A inside blood vessel 508, the cannula portion including four channels 505A and apertures 507A extending along the length of the portion and through which vessel blood flows. Blood vessel 508 and cannula 502A including channel 505A may be similar to that shown in FIG. 2A at 108, 102 including 105A, with exception of apertures 507A in channel 505A. Also shown is a central lumen 503A for transporting the IV fluid and/or guiding a needle, the central lumen including a cross-sectional geometry similar to that of the cannula portion; and four lumens 508A in which the vessel blood flows bordered by cannula 502A and a portion of a wall of vessel 508. Central lumen 503A and lumens 508A may be similar to that shown in FIG. 2A at 103 and 108A, except for apertures 507A.

In an exemplary embodiment of the present invention, FIG. 5E shows a cross-section of cannula 502A inside blood vessel 508, the cannula portion including one channel 505C and apertures 507A extending along the length of the portion and through which vessel blood flows. Blood vessel 508 and cannula 502A including channel 505C may be similar to that shown in FIG. 2C at 108, 102 including 105C, with exception of apertures 507A in channel 505C. Also shown is a central lumen 503A for transporting the IV fluid and/or guiding a needle, the central lumen including a cross-sectional geometry similar to that of the cannula portion; and one lumen 508C in which the vessel blood flows bordered by cannula 502A and a portion of a wall of vessel 508. Central lumen 503A and lumens 508A may be similar to that shown in FIG. 2C at 103 and 108C, except for apertures 507A.

In an exemplary embodiment of the present invention, FIG. 5E shows a cross-section of cannula 502A inside blood vessel 508, the cannula portion including three channels 505E and apertures 507A extending along the length of the portion and through which vessel blood flows. Blood vessel 508 and cannula 502A including channel 505E may be similar to that shown in FIG. 2E at 108, 102 including 105E, with exception of apertures 507A in channel 505E. Apertures 507A are shown in two channels 505C (optionally may be in all three channels or only in one). Also shown is a central lumen 503A for transporting the IV fluid and/or guiding a needle, the central lumen including a cross-sectional geometry which is circular; and three lumens 508E in which the vessel blood flows bordered by cannula 502A and the portion of the wall of vessel 508. Central lumen 503A and lumens 508E may be similar to that shown in FIG. 2E at 103 and 108E, except for apertures 507A.

The exemplary embodiments shown above in FIGS. 5D-5F are for illustrative purposes only, and are not intended to be limiting in any way. It should be evident to an ordinary person skilled in the art that there are numerous geometries which may be used for channeled cannula 502A, lumen 503A, channels 505A-505E, and lumens 508A-508E. Additionally, it should be evident that the distribution of a position of the channels in channel cannula 502 is for exemplary purposes only, and the there are numerous possibilities as to how the channels may be arranged. Similarly with respect to apertures 570 which may be accommodated in any arrangement in any number of channels, and optionally on other locations in the channel or on the cannula portion themselves.

Reference is now made to FIG. 5B which schematically illustrates an exemplary cannula portion 502B inserted through skin 510 into a blood vessel 508, and held in position by a wing 506B, according to some embodiments of the present invention. Cannula portion 502B includes a channel 505B for sufficiently maintaining a blood flow in vessel 508 while the cannula is inside the vessel. Cannula portion 502B additionally includes apertures 507B for delivering IV fluid into vessel 508 through multiple exit points in addition to a single exit point at a distal end 512B of the cannula, and for delivering IV fluid subcutaneously under skin 510. Optionally, the IV fluid is delivered only through apertures 507B. Optionally, the IV fluid is delivered only subcutaneously. Cannula portion 502B including channels 505B, apertures 507B, distal end 512B may be similar to that shown in FIG. 5A at 505A, 507A, and 512A. Blood vessel 508B may be similar to vessel 508A shown in FIG. 5A.

Reference is now made to FIG. 5C which schematically illustrates an exemplary cannula portion 502C inserted through skin 510 into a blood vessel 508, and held in position by a wing 506C, according to an embodiment of the present invention.

Cannula portion 502C includes a channel 515 formed internally in the cannula portion and configured to serve as an internal conduit for vessel blood to flow through while sufficiently maintaining the flow. Cannula portion 505C includes a at least one first opening 515A through which the vessel blood enters channel 515, and includes a second opening 515B in a distal section of the portion through which the blood flows out. Optionally, the vessel blood may flow out of channel 515 together with the IV fluid through an opening at distal end 515C. Optionally, second opening may be at distal end 515C and separate from the central lumen of cannula 502C. Cannula 502C may be substantially similar to cannula 102 in FIG. 1 with the difference that vessel blood flow is through channel 515 internally formed in the cannula portion.

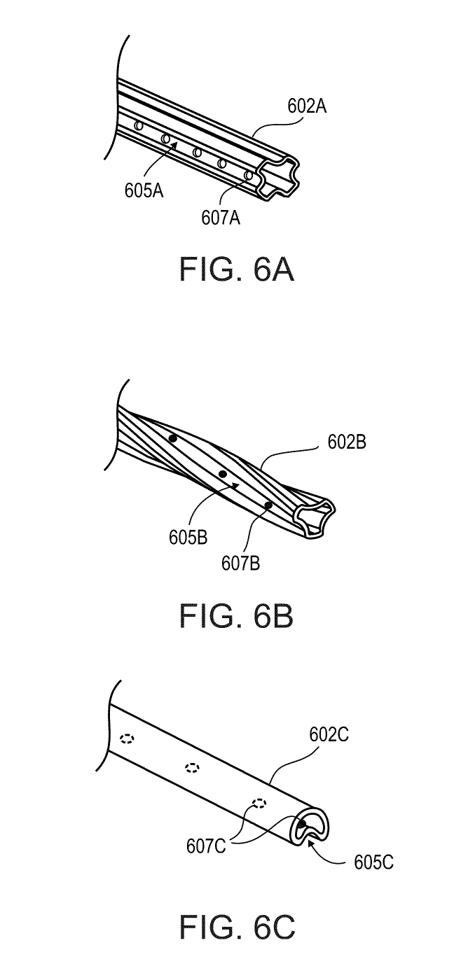

Reference is now made to FIG. 6A which schematically illustrates a perspective view of a cannula portion 602A including four channels 605A disposed in a straight line, according to some embodiments of the invention. Cannula portion 602A, including channel 605A, may be similar to that shown in FIG. 5D at 502A, including 505A. Optionally, cannula portion 502A may extend a whole length of the channeled cannula.

Reference is now made to FIG. 6B which schematically illustrates a perspective view of a cannula portion 602B including three channels 605B disposed in a helix, according to some embodiments of the invention. Cannula portion 602B, including channel 605B, may be similar to that shown in FIG. 5E at 502A, including 505C. Optionally, cannula portion 602B may extend a whole length of the channeled cannula.

Reference is now made to FIG. 6C which schematically illustrates a perspective view of a cannula portion 602C including a single channel 605C disposed in a straight line (crescent shaped cross-sectional channel), according to some embodiments of the invention. Cannula portion 602C, including channel 605C, may be similar to that shown in FIG. 5F at 502A, including 505E. Optionally, cannula portion 602C may extend a whole length of the channeled cannula.

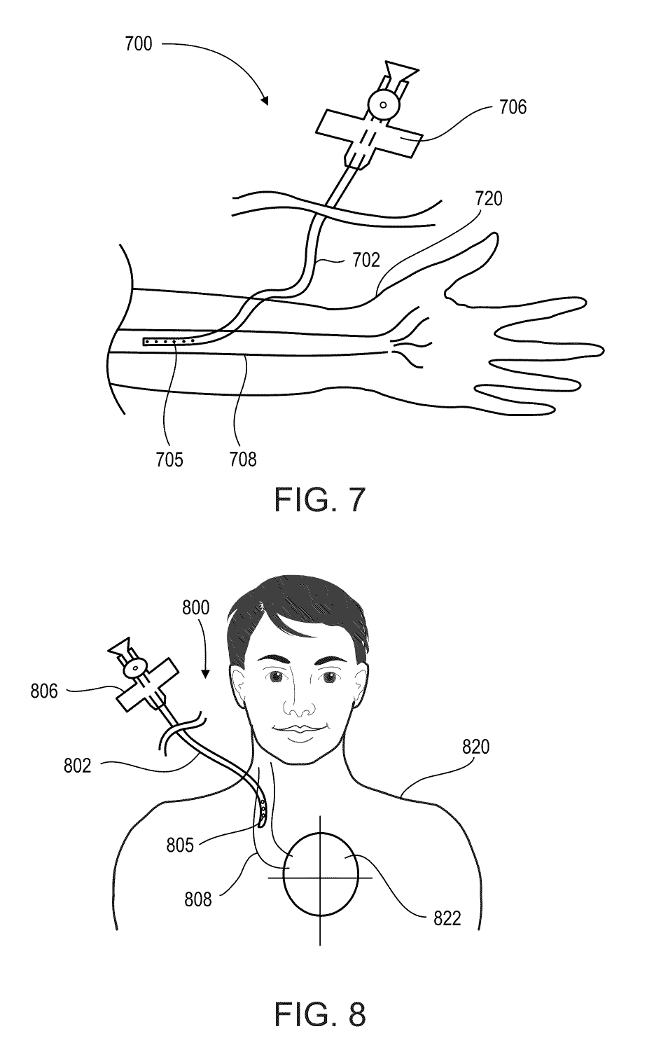

Reference is now made to FIG. 7 which schematically illustrates an IV cannulation device 700 including a channeled cannula 702 with apertures 705 used for peripheral venous access, according to some embodiments of the invention. Channeled cannula 702 includes a cannula portion at a distal end inserted in a blood vessel 708 in an arm 720 of a patient, and a proximal end attached to a hub 706. Hub 706 may be similar to hub 106 shown in FIG. 1. Channeled cannula 702 including apertures 705 may be similar to that shown in FIGS. 5A or 5B and/or 5D-5F. Optionally, device 700 may include a channeled cannula without apertures 507, similar to that shown in FIG. 1 and FIGS. 2A-2F at 102.

Reference is now made to FIG. 8 which schematically illustrates an IV cannulation device 800 including a channeled cannula 802 with apertures 805 used for central venous access, according to some embodiments of the invention. Channeled cannula 802 includes a cannula portion at a distal end inserted in a blood vessel 808, which may include a superior vena cava or an inferior vena cava of a patient for directly reaching into a right atrium of a heart 822 of a patient 820, and a proximal end attached to a hub 806. Hub 806 may be similar to hub 106 shown in FIG. 1. Channeled cannula 802 including apertures 805 may be similar to that shown in FIGS. 5A or 5B and/or 5D-5F. Optionally, device 800 may include a channeled cannula without apertures 807, similar to that shown in FIG. 1 and FIGS. 2A-2F at 102.

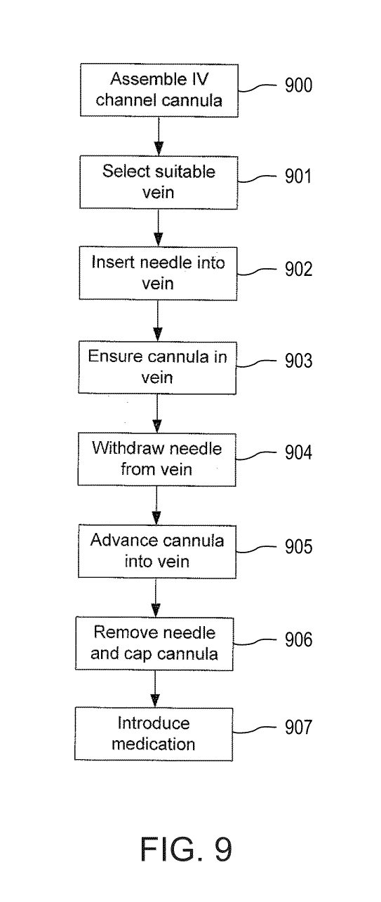

Reference is now made to FIG. 9 which illustrates a flow chart of a method for using exemplary cannula-over-needle device 100 including channeled cannula 102, according to an embodiment of the present invention. The method described is not intended to be limiting in any manner, and therefore, it may be evident to an ordinary person skilled in the art that there may be other ways of implementing the method. Furthermore, it may be possible to implement the method by varying and/or changing the steps, including their sequence as shown.

Optionally at 900, assemble device 100 by attaching a proximal end of channeled cannula 102 to hub 106. Optionally, channeled cannula 102 may include apertures for distributing the IV fluid over several exit points in blood vessel 108, similar to cannula 502A in FIG. 5A. Optionally, if subcutaneous distribution of the IV fluid is required, cannula 502B in FIG. 5B may be used. Assemble other components to hub 106 for example, flashback chamber, wings, valve, injection port, needle grip and needle 104, etc. Needle 104 is inserted into the proximal end of cannula 102 into lumen 103 and guided through the lumen out the distal end of the cannula.

Optionally at 901, a suitable blood vessel 108 (vein) is selected. If the IV cannulation includes peripheral venous access, a vein from the arm or the hand is selected. Optionally, from the leg or the foot. Optionally, for a neonatal, the vein may be selected from the head. If the IV cannulation includes central venous access, the superior vena cava or the inferior vena cava may be selected. The procedure may be assisted with imaging equipment such as ultrasound imaging for proper insertion of channeled cannula 102.

Optionally at 902, once blood vessel 108 is properly detected, needle 104 is inserted through skin 110 into the vessel.

Optionally at 903, once needle 104 is inside blood vessel 108, the needle may be advance several millimeters to ensure that channeled cannula 102 is inside the vessel.

Optionally at 904, once cannula 102 is properly inserted inside vessel 108, needle 104 is withdrawn from the blood vessel and retracted into lumen 103 by proximal pulling of the needle grip by the medical assistant (physician, nurse, or any medical personnel qualified to perform IV cannulation).

Optionally at 905, channeled cannula 102 is advanced inside vein 108 to the desired location.

Optionally at 906, physician extracts needle 104 from lumen 103 by proximally pulling on the needle grip. Once fully extracted, the medical assistant may place a cap on hub 106 where the needle grip was removed.

Optionally at 907, the medical assistant may administer the IV fluid into hub 106 for transporting by cannula 102 into the blood vessel. Channeled cannula 102 may first be flushed, for example by using a heparinized saline solution. Medication, when required, may be introduced combined with the IV fluid or may be separately injected into hub 106 through the injection port where it may be mixed with the IV fluid.

The terms “comprises”, “comprising”, “includes”, “including”, “having” and their conjugates mean “including but not limited to”. This term encompasses the terms “consisting of” and “consisting essentially of”.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, an and the include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. An intravenous (IV) cannula comprising;
an elongated body including a distal section having a fixed cross-section for insertion into a blood vessel; at least one channel extending along at least a portion of a longitudinal axis of a cannula section, the at least one channel configured to maintain an amount of at least 10% of a blood flow in the blood vessel; and a central lumen configured to allow an IV fluid flow into the blood vessel.

2. The IV cannula of claim 1 wherein the at least one channel is formed on an external surface of the cannula portion.

3. The IV cannula of claim 1 wherein the at least one channel forms at least one lumen between the cannula section and the blood vessel.

4. The IV cannula of claim 1 wherein a cross-sectional area of a channel is in a range between 0.02 mm²-2 mm² .

5. The IV cannula of claim 1 wherein a total cross-sectional area of the at least one channel is at least 10% of a cross-sectional area of said cannula section bounded by a smallest circle.

6. The IV cannula of claim 1 wherein a total cross-sectional area of the at least one channel is at least 10% of a cross-sectional area of said cannula section bounded by a smallest ellipse.

7. The IV cannula of claim 1 wherein the at least one channel is disposed in a straight line.

8. The IV cannula of claim 1 wherein the at least one channel is disposed in a helical configuration.

9. (canceled)

10. The IV cannula of claim 1 wherein a depth of the at least one channel is at least 1.2 times a maximum collapsible distance of a portion of a wall of the blood vessel.

11. The IV cannula of claim 1 wherein the at least one channel extends along a whole length of said IV cannula.

12-13. (canceled)

14. The IV cannula of claim 1 comprising apertures in said cannula section for allowing any one of, or any combination of, IV fluid and blood flow therethrough.

15. The IV cannula of claim 14 wherein the apertures are in the blood vessel.

16. The IV cannula of claim 14 wherein the apertures are in subcutaneous tissue.

17. (canceled)

18. The IV cannula of claim 1 wherein the channel is formed internally in said cannula.

19. (canceled)

20. The IV cannula of claim 1 wherein the at least one channel comprises 2 or more channels.

21-30. (canceled)

31. A method of IV cannulation comprising:
inserting an intravenous (IV) cannula having a distal section with a fixed cross-section into a blood vessel; administering an IV fluid into the lumen of the cannula; and
maintaining an amount of at least 10% of a blood flow in the blood vessel.

32. The method of claim 31 comprising allowing blood flow through at least one channel in the IV cannula.

33-36. (canceled)

37. The method of claim 31 comprising performing said IV cannulation in a peripheral vein or artery.

38. The method of claim 31 comprising performing said IV cannulation in a central vein or artery.

39-40. (canceled)

41. The method of claim 31 comprising maintaining an amount of at least 50% of a blood flow in the blood vessel.

42. (canceled)

43. The IV cannula of claim 1 wherein said fixed cross-section is axially variable.

44. The IV cannula of claim 1 wherein said blood flow is maintained when a portion of said blood vessel collapses onto the IV cannula.

45. An intravenous (IV) cannula comprising;
an elongated body including a distal section for insertion into a blood vessel;
at least one channel extending along at least a portion of a longitudinal axis of a cannula section prior to insertion into said blood vessel, the at least one channel configured to form a lumen with said blood vessel for blood flow therethrough; a central lumen configured to allow an IV fluid flow into the blood vessel.
